(19) United States
(12) Patent Application Publication
(10) Pub. No.: US 2016/0150766 A1
(43) Pub. Date: Jun. 2, 2016

(54) ADVANCING GENETIC GAIN IN BOVINE

(71) Applicants: Jennifer BARFIELD, Fort Collins, CO (US); Brandon L. WEBB, New Park, PA (US)

(72) Inventors: Jennifer BARFIELD, Fort Collins, CO (US); Brandon L. WEBB, New Park, PA (US)

(21) Appl. No.: 14/906,317

(22) PCT Filed: Jul. 23, 2014
(86) PCT No.: PCT/US14/47730

§ 371 Date: Jan. 20, 2016

Related U.S. Application Data
(60) Provisional application No. 61/857,359, filed on Jul. 23, 2013.

(51) Int. Cl. A01K 67/02; A61D 19/02; A61D 19/04; A61K 31/57; A61K 38/24

(52) U.S. Cl. CPC A01K 67/02; A61K 31/57; A61K 38/24; A61D 19/04; A61D 19/02; A01K 2227/101

(57) ABSTRACT

This invention relates to a method for reducing the generation interval and advancing genetic gain in bovines via pregnancies created by oocytes harvested from select prepubertal female calves less than six months of age and/or from smaller framed bovine breeds less than ten months of age, and alternatively less than nine months of age. Said animals are treated with a hormone regiment to advance the first pubertal estrus in order to retrieve oocytes for fertilization and implantation. Oocytes are collected from said animals via laparoscopic aspiration. In the case of oocytes that have not matured in vivo, the oocytes are transferred to and cultured to maturation. Once matured, oocytes are fertilized, and the resultant embryos are transferred to synchronized recipients.

### CROSS-REFERENCE TO RELATED APPLICATIONS

This application claims priority to U.S. Provisional Application Ser. No. 61/857,359 titled “Advancing Genetic Gain in Bovine,” filed on Jul. 23, 2013, which is incorporated herein, in its entirety, by this reference.

### FIELD OF THE INVENTION

The present invention relates to bovine reproduction and genetics. More specifically, the present invention advances the genetic gains of the most elite bovines in seedstock producing regions such as North America, Europe and Australia. Advancing genetic gain in animals, in turn, increases the genetic capacity of donor bovines and their offspring in those regions and ultimately in the developing world, resulting in increased milk and meat production levels and efficiencies within those populations.

### BACKGROUND OF THE INVENTION

Currently, in vitro fertilization (IVF) procedures are being conducted with bovines as young as 315 days of age and though that process is conducted sixty days sooner than artificial insemination or superovulation, the decrease in conception rates is nominal. Regardless of hormone levels and the ability to reproduce, current IVF procedures require rectal palpation in order to transvaginally aspirate oocytes, which is impossible with most dairy and beef breeds until at least 315 to 365 days of age.

Prepuberal Calves as Oocyte Donors: Promises and Problems, R. T. Duby, P. Damiani, C. R. Looney, R. A. Fissore and J. M Robl provides a target age of six to eight months of age. While this is considered prepubescent in cattle, it is not a substantial advantage when transvaginal aspirations are conducted as early as 10.5 months on larger framed breeds. As disclosed by Duby et al, in order to sensitize the calf follicles to later injections of follicle-stimulating hormone (FSH-P), which is used to induce superovulation, calves were injected with 250 i.u. of equine chorionic gonadotropin (eCG) hormone. However, this is not optimum as eCG is no longer considered legal for use in bovines within the United States or the European Union. This process would thus not be commercially viable.

In Duby et al., aspiration is accomplished through ultrasound-guided transvaginal probes. This can be problematic. Firstly, transvaginal probes cannot be used for aspiration of oocytes from calves less than six months of age regardless of the breed and most likely does not allow for the aspiration of oocytes from smaller framed breeds, such as Jersey, less than nine to ten months of age. Secondly, the lack of precision of transvaginal probes raises the risk of damage to the reproductive tract of the heifers. This could cause the heifers to be sterile and render them worthless. In addition, as disclosed by Duby et al., none of the oocytes collected from calves less than 240 days of age developed into pregnancies or even blastocysts.

There is therefore a continuing need for improved methods of shortening the generation interval in bovine. Not only is it desirable to increase the number of potential offspring from elite donors, but also the number of matings. As the science of genomics advances and as models for identifying optimal matings within certain breeds are further refined, increasing the number of matings per donor will allow the mating of female donors to sires with varying desirable genetic traits. For example, with the ability to mate an elite female donor an extra five times, one can target multiple mating sires that rank high not only for production, but also for health traits such as productive life, somatic cell count (SCC), and the like.

Additionally, as the science of genetically engineering bovines advances, so will the need to verify that the genes modified actually yield the anticipated result. There is thus a need for reducing the generation interval and time required for evaluation of subsequent generations from the present two years between the generations of genetically engineered beef and dairy-type bovines, as is the case with natural breeding and current commercially available IVF procedures.

There is, therefore, a need for a method providing acceleration of genetic gain in bovines via the creation of pregnancies from oocytes collected from prepubertal calves less than eight months of age via laparoscopy, and identifying female bovines within the population most likely to produce genetically superior offspring through mating optimization and the evaluation of genomic information.

### SUMMARY OF THE INVENTION

Through selective and corrective mating, the genetic capacity of bovines can be increased. This has been done in the cattle industry for decades. The industry has been limited in its genetic advancement due to the natural gestation length of 282 days and the fact that virgin females must be a minimum of 12-15 months of age prior to the initial mating. The invention, as disclosed herein, will allow the industry to move the initial mating to from 12-15 months to 3-6 months, thereby reducing the generation interval and allowing the industry to make more selective and corrective matings per elite donor while at the same time not risking the general or reproductive health of the donors.

It is an objective of the invention to accelerate genetic progress in all bovine dairy and beef breeds in order for those breeds to become more productive with longer productive lives, become more feed efficient, produce less methane and produce meat and milk that is of better quality and healthier for consumers.

It is a further objective of this invention to identify within beef and dairy cattle populations which of those currently highest ranked females and males will most likely produce the highest ranking offspring in the next generation.

It is a further objective of this invention to mate said individual female and male animals, which are most likely to produce the highest ranking offspring in the next generation, at the youngest possible ages, while at the same time maintaining the breeding integrity of the female donors.

It is a further objective to provide a target age of four to six months for at least one animal.

It is a further objective to provide a target age of three to four months for at least one animal, thereby shortening the generation interval to just 12-13 months.

It is a further objective of this invention to collect oocytes from prepubertal calves in a way that has no long-term negative impact on the reproductive or general health of the donor calves.

It is a further objective of this invention to provide a method that does not require the use of eCG hormone (equine chorionic gonadotropin).

It is a further objective of this invention to provide a method that uses chorionic gonadotropin-like (CG-like) protein.

It is a further objection of this invention to provide a method that uses gonadotropin-releasing hormone or an analog thereof.

It is a further objective of this invention to provide a method that uses FSH (follicle stimulating hormone).

It is a further objection of this invention to provide a method that uses LH (luteinizing hormone).

It is a further object of this invention to provide a method that uses LUTALYSE (Dinoprost Tromethamine) for the advancement of first pubertal estrus and/or to control timing of estrus and ovulation.

It is a further objective to provide a method that allows for aspiration of oocytes from calves less than six months of age and/or aspiration of oocytes from smaller framed breeds less than ten months of age, and alternatively less than nine months of age.

It is a further objective to provide a method having less chance of damaging the reproductive tract of the heifers as compared to aspiration with a traditional aspiration probe.

It is a further objective to provide a method that includes laparoscopic method.

It is a further objection of the present invention to provide a method including the use of a progesterone-releasing device, or other means of administering progesterone, to synchronize estrus in female bovines.

It is a further objection of the present invention to provide a method including a means of elevating progesterone through feeding, use of a progesterone-releasing device, injection, or other means of administering progesterone, to facilitate fixed-time artificial insemination.

It is a further objective of the present invention to provide a method including the use of a progesterone-releasing device to mature the reproductive tract prior to follicular aspiration.

It is a further objective of the present invention to provide a method including the use of a progesterone-releasing subcutaneous implantation to mature the reproductive tract prior to follicular aspiration.

It is a further objective of the present invention to provide a method including the use of progesterone-like feed additives, such as Melengestrol acetate (MGA), to mature the reproductive tract prior to follicular aspiration.

It is a further objective of the present invention to provide a method including the use of transdermal progesterone creams to mature the reproductive tract prior to follicular aspiration.

It is a further objective of the present invention to provide a method including the use of progesterone administration by injection or oral delivery to mature the reproductive tract prior to follicular aspiration.

It is a further objective of the present invention to provide a method having a hormone protocol and procedure that does not cause a negative impact on reproductivity.

It is a further objective of the present invention to improve fertility.

It is a further objective of the present invention to provide conception rates with artificial insemination post-procedure that meet or exceed the current industry average of 65 percent.

It is a further objective of the present invention to provide conception rates with artificial insemination post-procedure greater than 90 percent.

It is a further objective of the present invention to provide a method enabling six or more oocytes per procedure.

It is a further objective of the present invention to provide a method enabling eight or more oocytes per procedure.

It is a further objective of this invention to expand the genetic pool offered to beef and dairy cattle breeders and producers.

It is a further objective of this invention to expand the focus beyond the top one percent of females of various breeds in order to expand the genetic pool and minimize the problem of inbreeding.

It is a further objective of this invention to identify outcross pedigrees within different breeds, shorten the generation interval of the offspring of those outcrossed individuals, and accelerate the genetic progress of those bloodlines to further expand the genetic pool offered to beef and dairy cattle breeders and producers.

These and other objectives are achieved in the present invention.

There has thus been outlined, rather broadly, the more important features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

### DETAILED DESCRIPTION OF THE INVENTION

Laparoscopic oocyte retrieval in prepubescent calves younger than six months of age provides an avenue of shortening the generation interval, thus increasing the rate of genetic gain. To demonstrate the viability of this technology, oocytes were collected from superstimulated and non-superstimulated calves, each calf receiving each treatment at least once. Three laparoscopic oocyte retrievals were performed on each calf, and the oocytes collected were fertilized in vitro and cultured to the blastocyst stage of development. Resulting embryos were transferred fresh into synchronized recipients, and pregnancies were carried out to Day 60. On average, nine oocytes were retrieved per aspiration when the calves were superstimulated. An average of six oocytes were retrieved from calves that had no FSH treatment.

The first round of oocyte aspirations were conducted when the calves were 3.5 months of age. A total of 23 oocytes were collected from three calves, nine of those oocytes had compact cumulus, 11 had expanded cumulus, and three were denuded. The cleavage rate for all oocytes was 28% but none progressed to blastocyst stage. The second round of aspirations were conducted when the calves were 4.5 months of age. A total of 25 oocytes were collected and 24% cleaved. One embryo reached the blastocyst stage of development. It was transferred to a recipient but did not result in a pregnancy. The third round of aspirations were conducted when the calves were 5.5 months of age. A total of 24 oocytes were collected even though one calf did not respond to superstimulation. The overall cleavage rate was 73%, six embryos were produced, four were transferred to recipients, and two resulted in pregnancies that were sustained through 60 days of gestation. Both embryos that resulted in pregnancies were from calves that had been superstimulated.

Calves 3.5-6 months of age were used as egg donors. Initially a progesterone releasing device designed for sheep (Eazi Breed CIDR, Zoetis, USA) containing 0.3 g progesterone was inserted into the vagina of each calf and remained there for 12 days. The device was then removed for six days. A new CIDR was inserted and left for 12 days.

Superstimulation started nine days after insertion of the CIDR with the injection of eight doses follicle stimulating hormone (FSH) s.c. at 12-hour intervals, starting with two doses of 20 mg/dose, then two doses of 15 mg/dose, and finally two doses of 10 mg by the last injections. The CIDR was removed at the time of the final FSH injection and 50 mg of LH administered i.v. 2.5 days after removal of the CIDR. Oocyte aspiration via laparoscopy occurred 23 hours after LH administration.

On the day of oocytes aspiration, calves were led into an indoor facility with a manual squeeze chute adapted with a raised floor to accommodate the size of the calves. A belly band was used to hold the calves up in the event that they attempted to sit during the procedure. The area where the laparoscopic probe, camera, and aspiration needle were to be inserted was shaved and scrubbed with betadine followed by an ethanol rinse. The calf was given 0.25 mL Xylazine as a general sedative and approximately 20 mL Lidocaine was used as a local anesthetic at the sight of the incisions. Three incisions of approximately 1-1.5 inches were made for the laparoscopic probe, camera, and aspiration needle. Once the laparoscopic guides were inserted into the incisions, the body cavity was insufflated for easy of manipulation of the ovaries. Pincers were used to secure the position of the ovary and a needle guide was used to advance the needle forward. The camera allowed for visualization of the ovary and accurate aspiration of the follicles. The aspiration line was adapted to tubing that was connected to a 50 mL conical tube into which the oocytes were aspirated, which was connected to a vacuum pump set to 50 psi. Periodically, the aspiration line was rinsed with HCDM-M+5 mg/mL heparin to avoid blood clots in the lines in the collection tube. Both ovaries could be reached via the incisions made on the left side of the calf.

Once the aspiration was complete, the tube containing the aspirate was taken to the embryo lab and the aspirate rinsed through a filter to remove blood cells. Oocytes were isolated and put into a maturation medium as described below.

After the conclusion of one round of superstimulation/aspiration, calves were given two weeks to recover before being prepped for the next procedure as previously described beginning with the insertion of a CIDR.

The non-superstimulated calves underwent laparoscopic aspiration of oocytes without the use of hormones. These oocytes were immature upon collection and were matured in vitro. When hormones are not used, laparoscopic oocyte retrieval can be performed weekly but the interval between procedures will be longer if followed by a superstimulation aspiration.

Embryo generation was completed as follows:

Because the oocytes were not matured in vivo they were transferred to maturation medium for a period of time. Half of the oocytes from each aspiration were cultured for six or 12 hours, and then fertilized with frozen-thawed semen. Oocytes and spermatozoa were co-incubated for 18 hours, followed by removal of cumulus cells and culture in CDM-1 (chemically defined medium) for 56 hours. At the end of this incubation, embryos were checked for cleavage stage of development and those that had at least six cells were cultured further in CDM-2 for 96 hours.

Grade 1 and 2 morula or blastocyst stage embryos were transferred to synchronized recipients. Synchronization was done by two injections of PG 12 days apart with the second being given two days before aspiration of oocyte donors. Embryos were transferred fresh. All pregnancies were monitored until day 60 after which time the recipients were sold at auction. All three heifers conceived on the first service after completion of the procedures; that is, one hundred percent first service conception with artificial insemination.

In alternative embodiments LUTALYSE (Dinoprost Tromethamine) is used for the advancement of first pubertal estrus and/or to control timing of estrus and ovulation.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method for accelerating genetic gain in bovine animals, comprising:
selecting individual female and male bovine, wherein the female is at a prepubertal age;
treating the female with a hormone regimen, said hormone regimen comprising a combination of progesterone and prostaglandin, for a fixed time period;
collecting oocytes from said female;
fertilizing the oocytes with semen from said male; and
culturing the fertilized embryo.

2. The method of claim 1, further comprising transferring the embryo to a recipient animal.

3. The method of claim 1, wherein the female bovine is between four to six months of age.

4. The method of claim 1, wherein the female bovine is between three to four months of age.

5. The method of claim 1, wherein the method does not include use of equine chronic gonadotropin.

6. The method of claim 1, said hormone regimen including chorionic gonadotropin-like protein.

7. The method of claim 1, said hormone regimen including gonadotropin-releasing hormone or an analog thereof.

8. The method of claim 1, said hormone regimen including porcine follicle stimulating hormone.

9. The method of claim 1, said hormone regimen including luteinizing hormone.

10. The method of claim 1, wherein said treating the female with a hormone regimen includes administering progesterone by way of progesterone-like feed additives included in the female's food.

11. The method of claim 1, wherein the progesterone-like feed additives include melengestrol acetate (MGA).

12. The method of claim 1, wherein said treating the female with a hormone regimen includes administering progesterone by way of a progesterone releasing device, a subcutaneous implantation, injection, transdermal progesterone creams, or oral administration.

13. The method of claim 1, further comprising collecting at least six oocytes from said female.

14. The method of claim 1, further comprising collecting at least eight oocytes from said female.

15. The method of claim 1, wherein said method yields conception rates with said fertilization greater than 90 percent.

16. The method of claim 1, wherein the oocytes are collected via laparoscopy.

17. The method of claim 1, further comprising:
inserting a progesterone releasing device containing 0.3 grams progesterone into the vagina of said female;
administering porcine follicle stimulating hormone.

18. The method of claim 1 further comprising transferring the oocytes to maturation medium.
